(19) United States
(12) Patent Application Publication
(10) Pub. No.: US 2005/0149109 A1
(43) Pub. Date: Jul. 7, 2005

(54) Expanding filler coil

(76) Inventors: Michael P. Wallace, Fremont, CA (US); Joseph C. Eder, Los Altos Hills, CA (US); Jimmy D. Dao, Milpitas, CA (US)

Correspondence Address: ROBINS & PASTERNAK, 1731 EMBARCADERO ROAD, SUITE 230, PALO ALTO, CA, 94303, US

(21) Appl. No.: 10/745,911

(22) Filed: Dec. 23, 2003

(51) Int. Cl. A61M 29/00

(52) U.S. Cl. 606/200

(57) ABSTRACT

This is a device for occluding a space within the body. In particular, the device comprises an expandable member that has a restrained configuration and an expanded configuration and which assumes the expanded configuration upon deployment from a restraining member. The expandable member can be used alone or in combination with an inner member. The devices may be placed in a desired site within a mammal to facilitate the formation of an occlusion.

### FIELD OF THE INVENTION

Compositions and methods for repair of aneurysms are described. In particular, vaso-occlusive devices are disclosed, as are methods of making and using these devices.

### BACKGROUND

An aneurysm is a dilation of a blood vessel that poses a risk to health from the potential for rupture, clotting, or dissecting. Rupture of an aneurysm in the brain causes stroke, and rupture of an aneurysm in the abdomen causes shock. Cerebral aneurysms are usually detected in patients as the result of a seizure or hemorrhage and can result in significant morbidity or mortality.

There are a variety of materials and devices which have been used for treatment of aneurysms, including platinum and stainless steel microcoils, polyvinyl alcohol sponges (Ivalone), and other mechanical devices. For example, vaso-occlusion devices are surgical implements or implants that are placed within the vasculature of the human body, typically via a catheter, either to block the flow of blood through a vessel making up that portion of the vasculature through the formation of an embolus or to form such an embolus within an aneurysm stemming from the vessel. One widely used vaso-occlusive device is a helical wire coil having windings which may be dimensioned to engage the walls of the vessels. (See, e.g., U.S. Pat. No. 4,994,069 to Ritchart et al.) Other less stiff helically coiled devices have been described, as well as those involving woven braids. See, e.g., U.S. Pat. No. 6,299,627.

U.S. Pat. No. 5,354,295 and its parent, U.S. Pat. No. 5,122,136, both to Guglielmi et al., describe an electrolytically detachable embolic device. Vaso-occlusive coils having little or no inherent secondary shape have also been described. For instance, co-owned U.S. Pat. Nos. 5,690,666; 5,826,587; and 6,458,119 by Berenstein et al., describes coils having little or no shape after introduction into the vascular space. U.S. Pat. No. 5,382,259 describes non-expanding braids covering a primary coil structure.

In order to form an occlusion, multiple coils are typically packed into the aneurysm. Indeed, it has been shown that there is a positive correlation between the packing density of such devices (e.g., GDCs) and occlusion rate. See, e.g., Reul et al. (1997) Neurosurgery 41(5):1160-1165. Kawanbe et al. (2001) Acta Neurochir 143(5):451-455 report that packing densities in excess of 20% appear to be a critical factor in determining whether unwanted coil compaction will occur. To date, however, available coil designs can only be packed into aneurysms at densities of less than about 35% without risking aneurysm rupture. Piotin et al. (2000) AJNR 21:757-760.

None of these documents describe embolic compositions comprising an expandable element that is free from hydrogels that expands along its primary axis as described herein or methods of making and using such devices.

### SUMMARY OF THE INVENTION

Thus, this invention includes novel occlusive compositions as well as methods of using and making these compositions.

In certain aspects, the invention includes a vaso-occlusive device comprising a substantially tubular expandable member having a primary axis, wherein the expandable member expands along the primary axis upon release from a restraining member and further wherein the expandable member does not include (e.g., is free from) a hydrogel. In certain embodiments, the device further comprises an inner member that is surrounded by the expandable member. The inner and expandable members may be attached at one or more locations.

In any of the devices described herein, the expandable member may comprise a metal (e.g., nickel, titanium, platinum, gold, tungsten, iridium and alloys or combinations thereof such as nitinol) and/or a polymer (e.g., poly(ethyleneterephthalate), polypropylene, polyethylene, polyglycolic acid, polylactic acid, nylon, polyester, fluoropolymer, and copolymers or combinations thereof), for example one or more metal and/or polymer filaments in a braid configuration (e.g., a braid or one or more mono- and/or one or more multi-filaments).

Similarly, the inner member may comprise a metal (e.g., nickel, titanium, platinum, gold, tungsten, iridium, platinum, palladium, rhodium, gold, tungsten and alloys thereof including a stainless steel or super-elastic metal alloy such as nitinol) and/or a polymer (e.g., poly(ethyleneterephthalate), polypropylene, polyethylene, polyglycolic acid, polylactic acid, nylon, polyester, fluoropolymer, and copolymers or combinations thereof), for example a metal and/or polymer filament wound into a coil configuration. In certain embodiments, the inner member comprises a coil having a linear restrained configuration and a relaxed three-dimensional configuration, wherein the coil forms the relaxed three-dimensional configuration upon release from a restraining member.

Any of the devices described herein may further comprise a severable junction detachably which may be connected to a pusher element. The detachment junction can be positioned anywhere on the device, for example at one or both ends of the device. In certain embodiments, the severable junction(s) are, an electrolytically detachable assembly adapted to detach by imposition of a current; a mechanically detachable assembly adapted to detach by movement or pressure; a thermally detachable assembly adapted to detach by localized delivery of heat to the junction; a radiation detachable assembly adapted to detach by delivery of electromagnetic radiation to the junction or combinations thereof.

In certain embodiments, a detachment junction serves as the retraining member, for example when one or more ends of the expandable member secured to the detachment junction are released upon the imposition of current. In other embodiments, the restraining member may comprise a deployment catheter; a degradable polymer, a swellable polymer (e.g., hydrogel), a water-soluble coating; and/or combinations thereof. In any of the devices described herein, the restraining member may be removed upon deployment of the device or, alternatively, it may require additional actuation (e.g., change in external conditions such as temperature or ion concentration, application of energy, etc.) in order to remove the restraining member.

Furthermore, any of the devices described herein may further include an additional component, for example a polymer element and/or a bioactive coating such as one or more absorbable (biodegradable) polymers, for example, polyglycolide, poly-L-lactide, poly(g)-ethyl glutamates, polyphosphazene, polysaccharides, polyorthoesters, polycaprolactone, polyhydroxybutyrate, polydioxanone, polycarbonates, polyanhydrides, copolymers or blends thereof, collagen, elastin, fibrinogen, fibronectin, vitronectin, laminin, gelatin and combinations thereof. For example, a polymer may be positioned between the expandable member (and/or the inner member) and/or may be coated onto the expandable member (and/or the inner member).

In another aspect, the invention includes a method of occluding a body cavity comprising introducing any of the vaso-occlusive devices described herein into a body cavity (e.g., an aneurysm). ). In certain embodiments, the devices described herein are able to be packed into selected target site (e.g., aneurysms) at packing densities greater than about 35%. These and other embodiments of the subject invention will readily occur to those of skill in the art in light of the disclosure herein.

### BRIEF DESCRIPTION OF THE FIGURES

FIG. 1, panels A to C, are side-view cross-sections of an exemplary expandable vaso-occlusive device having an inner member (e.g., coil member) covered by an expandable member. FIG. 1A depicts the device as restrained (e.g., in a catheter) such that the diameter of the expandable member is restricted to the diameter of coil member diameter. Also shown are the detachment junction and pusher wire. FIG. 1B depicts the exemplary device shown in FIG. 1A immediately following deployment (detachment). At the proximal end of the device, where detachment occurs, the ends of the expandable member are no longer secured to the junction. FIG. 1C depicts the exemplary device of FIG. 1A and FIG. 1B as the unsecured ends of the expandable member expand along its primary axis.

FIG. 2, panels A to C, are side-view cross-sections of other exemplary vaso-occlusive devices having an inner member covered by an expandable member. In these embodiments, the expandable member does not continuously cover the inner member.

FIG. 3, panels A and B, depict side-view cross-sections of another exemplary expandable vaso-occlusive device having a constrained configuration prior to deployment (e.g., within the delivery catheter) and which expands along its primary axis upon deployment.

FIG. 4, panels A and B, are side-view cross-sections of another exemplary device having an inner member covered by an expandable member. FIG. 4A shows the device where the expandable member is constrained, for example within a catheter. FIG. 4B depicts how, upon deployment, the expandable member that is secured to the inner member expands.

FIG. 5, panels A and B, are side-view cross-section depictions of an exemplary device in which the expandable member is secured to the inner member in multiple locations. FIG. 5A shows the device where the expandable member is constrained in a restraining member (e.g., catheter), for example within a catheter. FIG. 5B depicts how, upon deployment, the expandable member that is secured to the inner member in multiple locations expands.

FIG. 6, panels A to D, are side-view cross-section depictions of exemplary devices including an expandable member, an inner member and an additional polymer layer. FIG. 6A depicts the polymer encasing the expandable member on both of its external and internal faces. FIG. 6B depicts the polymer when positioned between the expandable member and the inner member. FIG. 6C depicts the polymer element surrounding the expandable member on its external face. FIG. 6D depicts the polymer coating the inner member. In FIGS. 6A-D, the polymer may be secured or unsecured to one or more of the inner member and/or expandable member.

FIG. 7, panels A to C, are side view depictions of exemplary expandable members comprising braid (700) and coil (710) constructions. Also shown in the Figures is the inner member (702) surrounded by the expandable member. FIG. 7A shows a construction in which the coil (710) constructions are positioned near the ends of the expandable member. The transition from coil to braid is shown (715). FIG. 7B depicts an alternating coil (710) and braid (700) construction. FIG. 7C depicts a construction in which the coil (710) constructions are used to secure the expandable member to the inner member (702).

FIG. 8, panels A and B, are side view depictions of exemplary devices as described herein comprising expandable member (800); and inner coil member (810). Expandable member (800) is made by winding braid into a coil-like structure. FIG. 8A shows the restrained configuration. FIG. 8B shows expansion of expandable member after release from a restraining member.

### DESCRIPTION OF THE INVENTION

Occlusive (e.g., embolic) compositions are described. The compositions described herein find use in vascular and neurovascular indications and are particularly useful in treating aneurysms, for example small-diameter, curved or otherwise difficult to access vasculature, for example aneurysms, such as cerebral aneurysms. Methods of making and using these vaso-occlusive elements also an aspects of this invention. The compositions and methods described herein may achieve better occlusion and treatment outcomes than known devices, for example because they can achieve higher packing densities and/or because the expandable nature means that fewer devices are required to more fully occlude a vessel.

Advantages of the present invention include, but are not limited to, (i) the provision of expandable occlusive (embolic) devices; (ii) the provision of occlusive expandable devices that more quickly fill the selected site; (iii) the provision of occlusive elements that can be packed into aneurysms at high densities; (iv) the provision of occlusive devices that can be retrieved and/or repositioned after deployment; and (v) cost-effective production of these devices.

All publications, patents and patent applications cited herein, whether above or below, are hereby incorporated by reference in their entirety.

It must be noted that, as used in this specification and the appended claims, the singular forms “a”, “an”, and “the” include plural referents unless the content clearly dictates otherwise. Thus, for example, reference to a device comprising “an expandable member” includes devices comprising of two or more expandable members.

General Overview

Described herein are novel vaso-occlusive elements and methods of manufacturing these elements. Unlike previously described vaso-occlusive elements, the devices described herein include at least one element that expands along its primary axis after removal from a restraining member. In addition, unlike previously described devices, the devices described herein can be packed into aneurysms at high densities, typically greater than about 35%-45% (or any value therebetween), perferably greater than about 40%-50% (or any value therebetween), and even more preferably greater than about 50%.

The devices described herein comprise at least one expandable element. In certain aspects, the devices comprise an expandable member in combination with an inner member. The expandable member is typically free of any hydrogels (substances that are gels in an aqueous environment and expand to at least 10 times their original size upon contact with water), but may include one or more non-hydrogel polymers that do expand.

The expandable member may assume a variety of tubular structures, for examples, braids, coil, combination braid and coils and the like. Similarly, although depicted in the Figures described below as a coil, the inner member may be of a variety of shapes or configuration includes, but not limited to, braids, knits, woven structures, tubes (e.g., perforated or slotted tubes), injection-molded devices and the like. See, e.g., U.S. Pat. No. 6,533,801 and International Patent Publication WO 02/096273. The inner member may also change shape upon release from the restraining member, for example change from a constrained linear form to a relaxed, three-dimensional configuration upon deployment. However, the inner member does not expand along its primary axis in the sense of the expandable member.

Materials

The expandable member and/or inner member may be made of any material or combination of materials, including for example, metals, polymers (e.g., natural or synthetic polymers), absorbable (degradable) materials, filamentary materials such as Dacron, cotton, or other materials or combinations thereof. Unlike known devices, the expandable members described herein are free from hydrophobic hydrogels (foams) that expand in contact with water. See, e.g., U.S. Pat. No. 6,500,190. The expandable member may, however, include non-hydrogel polymers that are capable of expansion.

Suitable metals and alloys for the wire include the Platinum Group metals, especially platinum, rhodium, palladium, rhenium, as well as tungsten, gold, silver, tantalum, and alloys of these metals. The expandable member and/or inner member may also comprise of any of a wide variety of stainless steels if some sacrifice of radio-opacity may be tolerated. Very desirable materials of construction, from a mechanical point of view, are materials that maintain their shape despite being subjected to high stress. Certain “super-elastic alloys” include nickel/titanium alloys (48-58 atomic % nickel and optionally containing modest amounts of iron); copper/zinc alloys (38-42 weight % zinc); copper/zinc alloys containing 1-10 weight % of beryllium, silicon, tin, aluminum, or gallium; or nickel/aluminum alloys (36-38 atomic % aluminum). Particularly preferred are the alloys described in U.S. Pat. Nos. 3,174,851; 3,351,463; and 3,753,700. Especially preferred is the titanium/nickel alloy known as “nitinol.” These are very sturdy alloys that will tolerate significant flexing without deformation even when used as a very small diameter wire. If a super-elastic alloy such as nitinol is used in any component of the device, the diameter of the wire may be significantly smaller than that used when the relatively more ductile platinum or platinum/tungsten alloy is used as the material of construction. These metals have significant radio-opacity and in their alloys may be tailored to accomplish an appropriate blend of flexibility and stiffness. They are also largely biologically inert. Highly preferred is a platinum/tungsten alloy.

The expandable member and/or inner member may comprise one or more polymers. Non-limiting examples of polymers and/or biodegradable (absorbable) materials including, for example, lactide, glycolide, and caprolactone polymers and their copolymers; hydroxybutyrate and polyhydroxyvalerate and their block and random copolymers; a polyether ester; anhydrides, polymers and copolymers of sebacic acid, hexadecandioic acid; orthoesters and combinations thereof. See, e.g., U.S. Pat. No. 6,585,754 and 6,280,457. The expandable and/or inner members may also be combinations of one or more metals and one or more polymers.

In certain embodiments, the expandable member comprises a material having shape-memory properties (e.g., super-elastic properties), including materials such as nitinol (see, above) and/or shape memory polymers such as those described in International Publication WO 03/51444.

One or both of the inner and expandable members may also comprise additional components (described in further detail below), such as co-solvents, plasticizers, coalescing solvents, bioactive agents, antimicrobial agents, antithrombogenic agents, antibiotics, pigments, radiopacifiers and/or ion conductors which may be coated using any suitable method or may be incorporated into the element(s) during production.

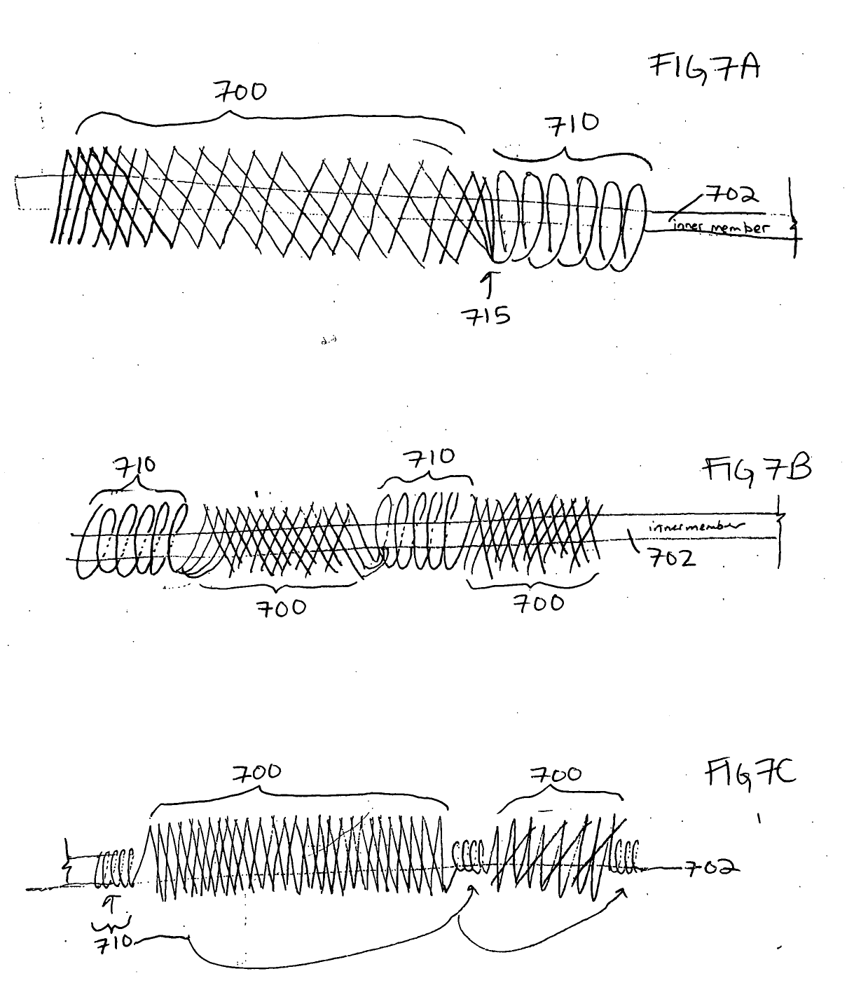

As noted above, the expandable element may take many forms including, for example, a braid, a coil-like structure, combination braid-coils, or any other substantially tubular structure that expands. See, also, FIG. 7A to 7C. The overall structure of the expandable member is preferably tubular, although as shown in the drawings, the diameter of this element is not necessarily constant along its length. By “expandable,” is meant that the element increases in diameter along its primary axis. This is to be contrasted with many known vaso-occlusive devices (described above), which may change configuration (e.g., from a constrained linear configuration to a relaxed three-dimensional configuration) but do not expand along their primary axis. Expansion refers to any increase in diameter along the primary axis and may be for example, an expansion from approximately 0.12 mm to about 20 mm (or any value therebetween), more preferably from about 0.25 mm to about 3 mm (or any value therebetween), even more preferably from about 0.5 mm to about 2 mm (or any value therebetween).

Restraining Members

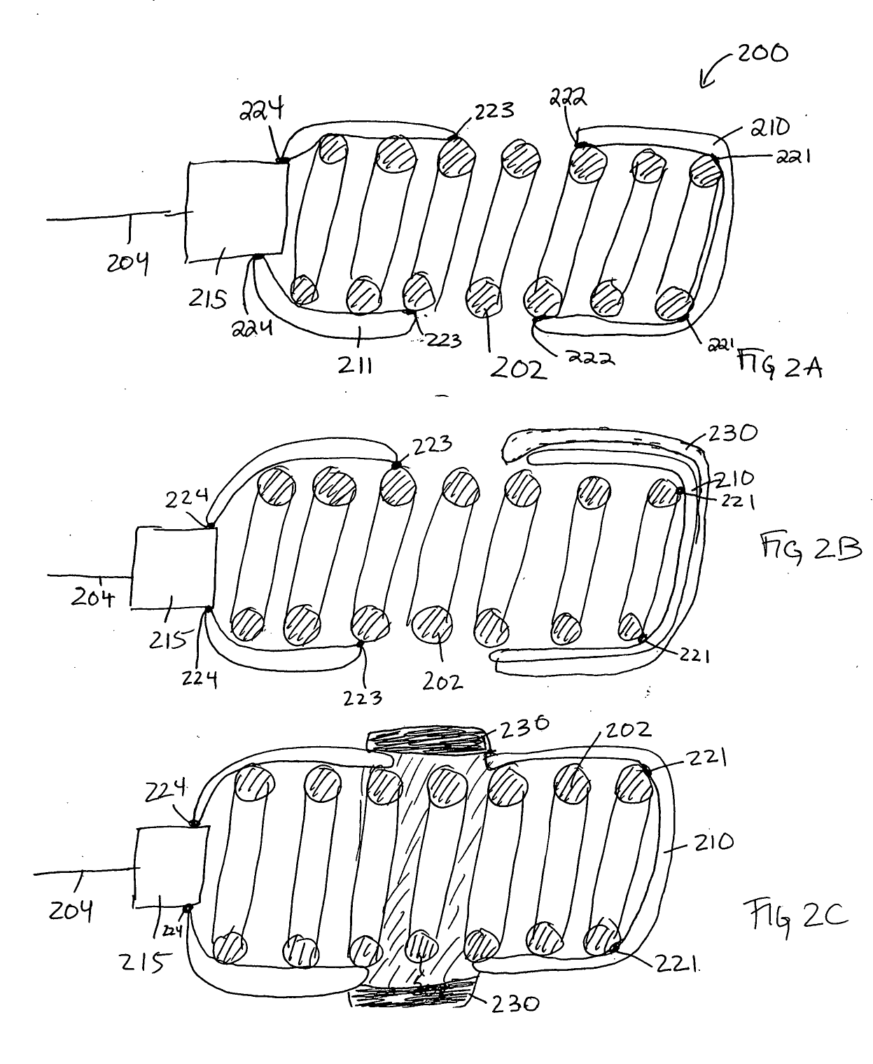

The expandable member typically self-expands upon release from a restraining member (e.g., following deployment and/or detachment) or shortly after release from a restraining member. Exemplary restraining elements are described below and depicted in FIG. 2A-C. Non-limiting examples of restraining members include catheters or other devices that physically restrain the expandable member's expansion; attachment of one or more regions of the expandable member (e.g., ends) to one or more detachment junctions (e.g., electrolytically detachable junctions) such that the expandable member can expand after detachment; degradable or swellable polymers; one or more water-soluble substances (e.g., as coatings); removable physical members (e.g., rings) that restrain the expandable member from expanding until they are degraded or allowed to swell; and/or differences in external conditions after deployment that induce expansion (e.g., body temperature and/or exposure to an aqueous environment may induce expansion of shape-memory materials).

In certain embodiments, the restraining member comprises a deployment catheter (and/or detachment junction) and the expandable member expands upon release from the catheter and/or detachment junction. For example, the non-hydrogel expandable member may relax immediately upon removal of the restraining catheter and/or detachment from the detachment junction. Alternatively, the non-hydrogel expandable member may assume its expanded configuration upon exposure to an aqueous environment such as blood (e.g., by virtue of the lower surface tension in the aqueous environment).

In other embodiments, the restraining member comprises a coating of a material (or combination of materials) that swells, dissolves, degrades over time and/or is removed by application of energy or the like.

Non-limiting examples of suitable swellable polymers for use as restraining members include hydrogels, which are capable of absorbing a desired amount of aqueous fluid. Examples of swellable polymers include materials formed from homopolymers, copolymers, and/or network polymers containing: polyethylene glycol, polypropylene glycol, polyvinyl alcohol, polyvinylpyrrolidone, polyacrylates, polymethacrylates, polyacrylamides, polyethyloxazoline, polysaccharides, mucopolysaccharides, polyaminoacids, carboxy alkyl celluloses, partially oxidized cellulose, hyaluronic acid, dextran, heparin sulfate, chondroitin sulfate, heparin, agar, starch, alginate, fibronectin, gelatin, collagen, fibrin, pectins, albumin, polyesters of alpha-hydroxy acids, polyglycolic acid, poly-DL-lactic, poly-L-lactic acid, polylactones, polyanhydrides, polyorthoesters, polydioxanone, polycaprolactones, poly(delta-valerolactone), poly(gamma-butyrolactone), and combinations thereof. The gel may further comprise a chemical cross-linking agent having two or more reactive groups in order to form chemical bridges between two or more polymeric molecules. Examples of such cross-linking agents include diacrylates, oligoacrylates, dimethacrylates, oligomethacrylates, divinyl ethers, certain cations, and combinations thereof.

The terms “absorbable” and “biodegradable” are used interchangeable to refer to any agent that, over time, is no longer identifiable at the site of application in the form it was injected, for example having been removed via degradation, metabolism, dissolving or any passive or active removal procedure. In preferred embodiments, the polymer comprises, for example, polyglycolide, poly-L-lactide, poly(g)-ethyl glutamates, polyphosphazene, polysaccharides, polyorthoesters, polycaprolactone, polyhydroxybutyrate, polydioxanone, polycarbonates, polyanhydrides, copolymers of one of these polymers and/or blends of these polymers. Non-limiting examples of bioabsorbable proteins include synthetic and polysaccharide biodegradable hydrogels, collagen, elastin, fibrinogen, fibronectin, vitronectin, laminin and gelatin. Many of these materials are commercially available. Fibrin-containing compositions are commercially available, for example from Baxter. Collagen containing compositions are commercially available, for example from Cohesion Technologies, Inc., Palo Alto, Calif. Fibrinogen-containing compositions are described, for example, in U.S. Pat. Nos. 6,168,788 and 5,290,552. As will be readily apparent, absorbable materials can be used alone or in any combination with each other and/or other materials.

Non-limiting examples of water-soluble materials that may be used as restraining members are described in co-owned U.S. Pat. Nos. 5,980,550 and 6,299,627 and include, poly vinyl alcohol, polyvinylpyrolidone, starch (polysaccharides), polypropolene oxide (PPO), polyethylene oxide (PEO), and/or PPO-PEO co-polymers (pluronics). Generally, these materials dissolve anywhere between 1 second and 20 minutes (or any time therebetween), preferably between about 5 and 10 minutes after deployment. As will be apparent, the water-soluble coating is placed on a sufficient amount of the device to inhibit expansion of the expandable member prior to deployment. Multiple water-soluble materials and/or coatings may be employed. For example, one coating may inhibit expansion while another dissolvable coating that resists thrombis formation may be used so that the device can be more stable in the vessel before thrombosis formation begins (e.g., a anti-thrombogenic coating that dissolves in a couple of hours or so).

In certain instances, additional actuation may be needed in order remove the restraining member. Non-limiting examples of such actuation include inducing changes in external conditions such as temperature changes, adjustment of the concentration of the surrounding medium (e.g., ions, etc.), application of energy (e.g., light, electrical, etc.) and the like. For instance, an expandable member comprising a shape-memory material (e.g., nitinol or shape-memory polymer) may require the additional actuation of a warm (above body temperature) bolus to the vessel to induce expansion. Similarly, the imposition of an electrical current may be used to detach a restraining member that surrounds the expandable member (see, also FIG. 2C). As will be apparent, the additional actuation should not be harmful to the subject.

Thus, the restraining members may be removed by any suitable mechanism as described herein including degradation, swelling, electrolytic detachment, activation by light, temperature of the like.

### EXEMPLARY EMBODIMENTS

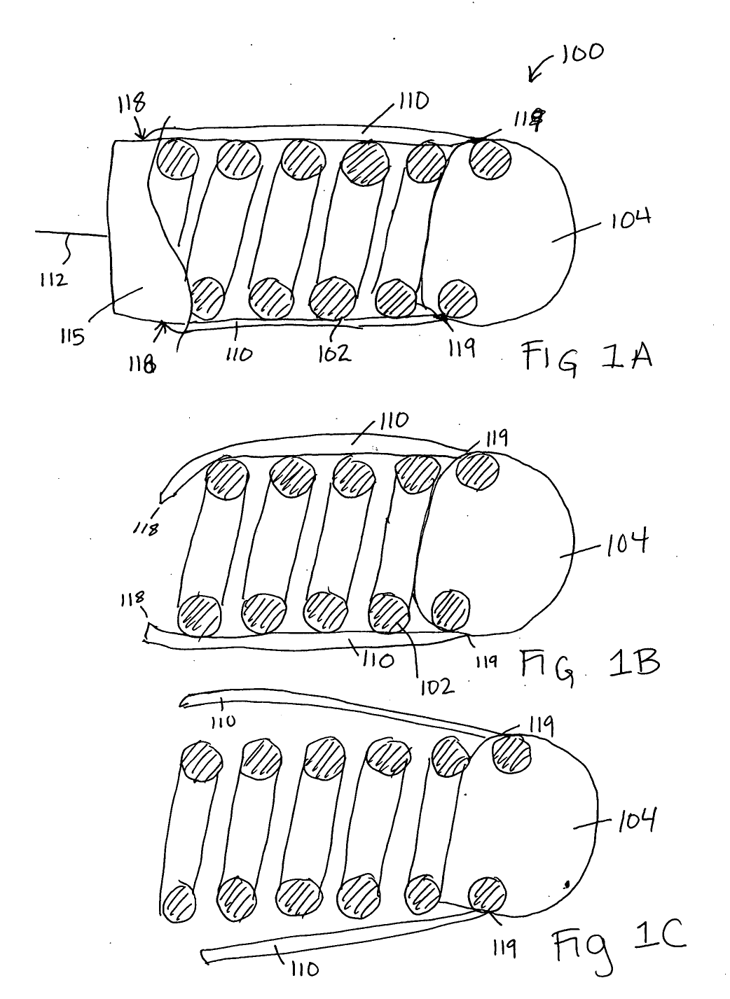

FIG. 1A-C show cross-sections of an exemplary device (100) according to this disclosure. These variations are made up of an inner coil member (102), depicted in a helical form, and an expandable member (110) surrounding the inner member (102). Also shown in FIG. 1A. are pusher wire (112), detachment junction (115) and termination end (104).

In the variation shown in FIG. 1A, the expandable member (110) is secured at one end (118) to the detachment junction (115) and at the other end (119) to the distal end of the inner member (102). The inner member (102) is covered with expandable member (110). The expandable member (110) is typically substantially tubular in shape. When the ends of the expandable member are restrained, for example to a detachment junction and/or by a deployment mechanism, the diameter of the substantially tubular shape is not constant along its length and may assume an elongated oval shape.

When restrained (e.g., by attachment of one or more of its ends to a detachment junction and/or by a deployment catheter), the diameter of the expandable member (110) is essentially that of the linear coil member (102). Upon deployment (e.g., detachment from the junction and/or removal of restraining catheter), the expandable member expands (e.g., the distance across the member (diameter) gets larger). The expandable member may be self-expanding upon deployment and/or may be induced to expand after deployment using any suitable mechanism. For example, in certain embodiments, the inner member may assume a relaxed configuration upon deployment, and the self-formation of the inner member into a three-dimensional relaxed configuration may induce expansion of the surrounding expandable member.

In embodiments using a guide wire, the guide wire typically has a diameter of 0.010 to 0.020 inches diameter in the portion more proximal of the portion shown in the drawing. The guide wire may be joined at one or more locations to the device, for example by soldering or the like. The devices are detached from the guide wire by electrolytic decomposition of either the joints or some section of the guide wire. The current level may be altered or the flow of conductive fluids such as saline solution may be introduced to enhance the electrolysis rate.

FIG. 1B shows the device of FIG. 1A shortly following deployment from the catheter and detachment from the detachment junction. The ends of the expandable member (110) that were formerly secured to the detachment junction are no longer secured and the expandable member (110) begins to expand along its primary axis. FIG. 1C shows the same device at a later time point and demonstrate how the detached ends of the expandable member (110) allow expansion of this element. The inner coil member (102) may also change configuration. FIG. 2A-C show side-view cross-sections of other exemplary devices (200) according to the present disclosure. In the embodiments depicted in FIG. 2A-C, expandable member (210, 211) is not a single element but, rather, comprises two or more distinct elements (210, 211) covering different regions of the inner coil member (202). It is to be understood that in embodiments comprising multiple expandable members in combination with inner members, the expandable members may cover the entire inner coil member (e.g., overlapping each other or non-overlapping) or less than the entire inner member, as depicted in FIG. 2A-C.

FIG. 2A is a side view of an expandable member-covered (201, 211) inner member (202) device according to the invention similar in make-up and size as the devices shown in FIG. 1A-1C. FIG. 2A also shows pusher wire (204), detachment junction (215). Expandable member (210) is optionally secured to the coil as shown (221, 222). Expandable member (211) is optionally secured to at one end (223) at or near the distal end of the inner coil member (202) and at its other end (224) to detachment junction (215). Although depicted as attached, it is to be understood that expandable members (210, 211) may be unattached to the inner coil member or secured to the coil member in one or more locations.

FIG. 2B shows a multi-expandable member design similar to FIG. 2A in which less than all ends of one of the expandable elements (210) are not attached to the inner coil member. FIG. 2B shows expandable members (201, 211), inner coil member (202), pusher wire (204), detachment junction (215). One expandable member (210) is secured to the coil as shown (221). Another expandable member (211) is secured by one of its ends (223) to the distal portion of the inner member (202) and by its other end (224) to detachment junction (215). Restraining member (230) covers expandable member (210), thereby restrained the unsecured ends (225) of expandable member (210) prior to deployment. It should be noted that one or more of the unsecured ends (225) might also be secured to the inner member. Restraining member (230) may comprise a biodegradable material; water-soluble material and/or swellable material. Restraining member (230) may also extend to cover additional expandable members. Upon deployment, the restraining member (230) degrades or enlarges, thereby allowing expansion of expandable members.

FIG. 2C shows a design in which some or all of the discontinuous expandable members (e.g., the unsecured ends) are restrained by a detachable material (e.g., an electrolytically detachable ring structure). FIG. 2C shows expandable members (210, 211), inner coil member (202), pusher wire (204), detachment junction (215). Expandable member (210) is secured to the coil as shown (221). Expandable member (211) is secured proximally (224) to detachment junction (215). Restraining member (230) covers expandable members (210, 211), thereby restrained the unsecured ends prior to deployment. Restraining member (230) may comprise a detachable structure such as an electrolytically detachable ring. Upon deployment and detachment of the restraining member (230), the diameter of the expandable members expands and fills the aneurysm.

Thus, in embodiments such as those shown in FIG. 2B-C (e.g., which one or more expandable members or regions thereof (e.g., ends) are not attached to the inner coil member), it is preferred that some or all of the expandable member may be constrained by another material, for example a biodegradable (absorbable) material, a water-soluble coating, a detachable mechanical device (e.g., electrolytically detachable structure such as a ring), and/or by a swellable polymer. Upon removal of the restraining member (e.g., degradation of the absorbable material, dissolving of the water-soluble material, detachment of the structure and/or swelling of the polymer), the expandable member expands, allowing in filling of the aneurysm.

It is to be understood that the disclosure herein encompasses multiple-expandable members in which each expandable member is secured (at one or more regions) to the coil member, for example at the end of each expandable member. In addition, one or more of the features of the embodiments described herein may be combined in a single device, for example a device comprising a discontinuous expandable member, wherein one of the expandable members is secured to the detachment junction and other expandable members are restrained by swellable polymers, biodegradable substances and/or one or more restraining mechanical elements, such as rings or the like.

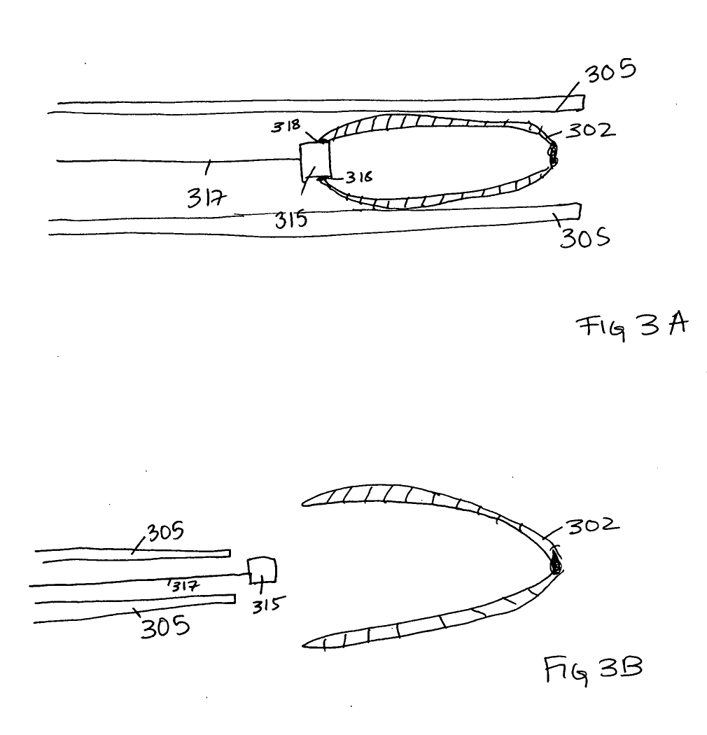

FIG. 3A and 3B are side view cross-sections of an exemplary embodiment that does not include an inner member. FIG. 3A shows expandable member (302) secured (318) to (and restrained by) detachment junction (315) on pusher wire (317). When expandable member (302) is restrained in deployment mechanism (305), for instance a catheter, the diameter is restricted. FIG. 3B depicts that, upon detachment, the ends of the expandable member (302) are no longer secured to the detachment junction (315) and the device expands.

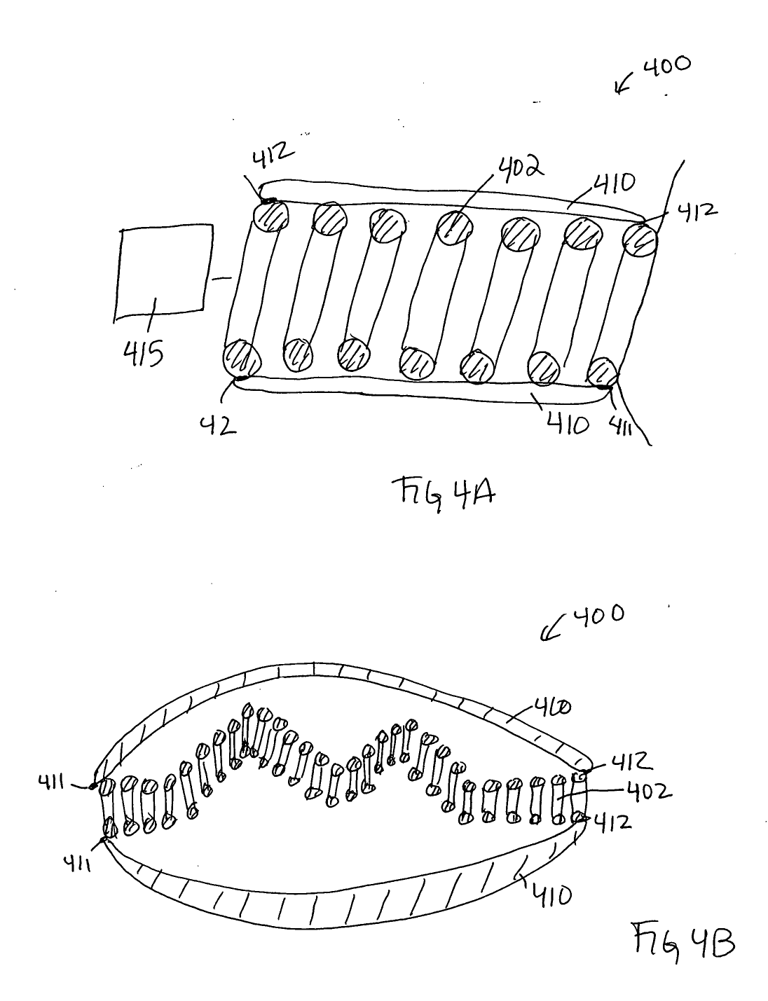

FIG. 4A and 4B depict exemplary devices similar to those described above comprising an inner coil member (402) to which an expandable element (410) is secured by its ends at or near the ends of the coil member. FIG. 4A depicts the device as constrained, for example within a restraining member (deployment catheter), including inner member (402); expandable member (410); detachment junction (415); and attachment points (411, 412) of expandable member (410) to ends of coil member (402). It should be noted that although FIG. 4A the expandable member (410) may also be secured to (restrained by) the detachment junction (415), and released upon detachment. FIG. 4B is a side view depicting expansion of the expandable member (410) upon deployment, causing the coil member (402) to compress, shorten and/or buckle.

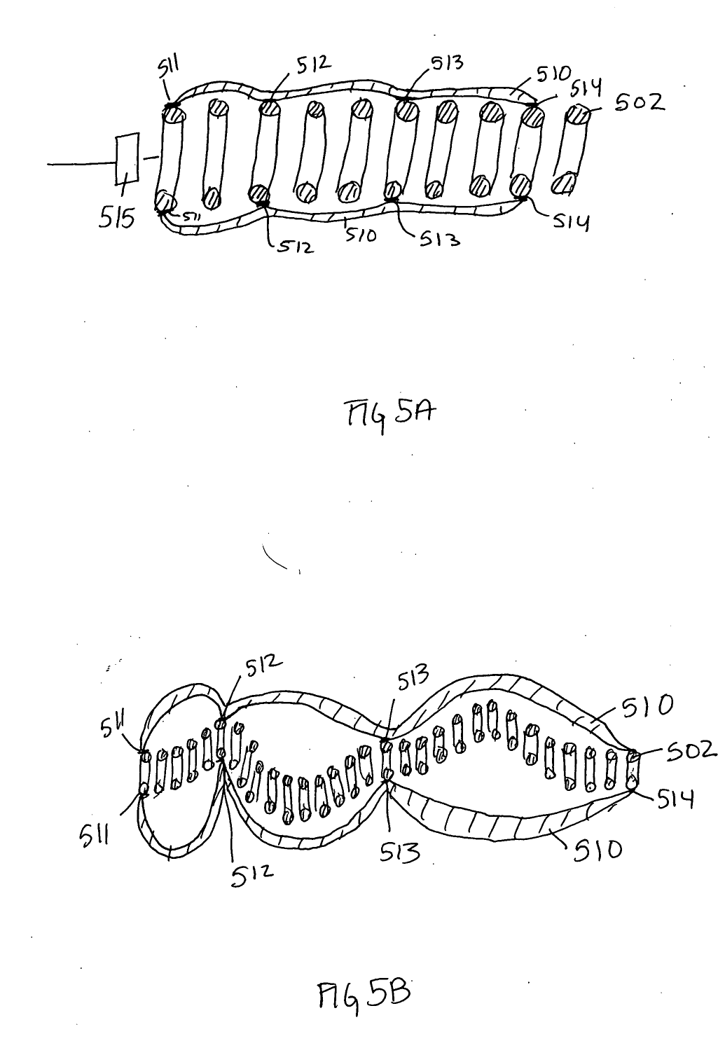

FIG. 5A and 5B depict exemplary devices similar to FIG. 4A and 4B. FIG. 5A depicts the device as restrained, for example within a deployment catheter, including coil member (502); expandable member (510); detachment junction (515); and multiple attachment points (511, 512, 513, 514) of expandable member (510) to multiple locations of coil member (502). The expandable member may be secured to the detachment junction (515) and released upon detachment. FIG. 5B is a side view cross-section depicting expansion of the expandable member (510) upon deployment, causing the coil member (502) to compress, shorten and/or buckle, particularly where it is attached to the coil.

Any of the devices described herein may also be retrievable and/or re-positioned after deployment using systems and methods known to those working in the field, for example as described in U.S. Pat. Nos. 5,868,754; 5,382,259; and WO 03/026487.

Additional Components

Furthermore, the vaso-occlusive devices of the present invention can also be used in combination with additional components. For example, lubricious materials (e.g., hydrophilic) materials may be used to coat one or more members of the device to help facilitate delivery. Cyanoacrylate resins (particularly n-butylcyanoacrylate), particular embolization materials such as microparticles of polyvinyl alcohol foam may also be introduced into the intended site after the inventive devices are in place.

One or more bioactive materials may also be included. See, e.g., co-owned U.S. Pat. No. 6,585,754 and WO 02/051460. The term “bioactive” refers to any agent that exhibits effects in vivo, for example a thrombotic agent, an anti-thrombotic agent (e.g., a water-soluble agent that inhibits thrombosis for a limited time period, described above), a therapeutic agent (e.g., chemotherapeutic agent) or the like. Non-limiting examples of bioactive materials include cytokines; extracellular matrix molecules (e.g., collagen); trace metals (e.g., copper); and other molecules that stabilize thrombus formation or inhibit clot lysis (e.g., proteins or functional fragments of proteins, including but not limited to Factor XIII, α₂-antiplasmin, plasminogen activator inhibitor-1 (PAI-1) or the like). Non-limiting examples of cytokines which may be used alone or in combination in the practice of the present invention include, basic fibroblast growth factor (bFGF), platelet derived growth factor (PDGF), vascular endothelial growth factor (VEGF), transforming growth factor beta (TGF-β) and the like. Cytokines, extracellular matrix molecules and thrombus stabilizing molecules (e.g., Factor XIII, PAI-1, etc.) are commercially available from several vendors such as, for example, Genzyme (Framingham, Mass.), Genentech (South San Francisco, Calif.), Amgen (Thousand Oaks, Calif.), R&D Systems and Immunex (Seattle, Wash.). Additionally, bioactive polypeptides can be synthesized recombinantly as the sequence of many of these molecules are also available, for example, from the GenBank database. Thus, it is intended that the invention include use of DNA or RNA encoding any of the bioactive molecules. Cells (e.g., fibroblasts, stem cells, etc.) can also be included. Such cells may be genetically modified. Furthermore, it is intended, although not always explicitly stated, that molecules having similar biological activity as wild-type or purified cytokines, extracellular matrix molecules and thrombus-stabilizing proteins (e.g., recombinantly produced or mutants thereof) and nucleic acid encoding these molecules are intended to be used within the spirit and scope of the invention. Further, the amount and concentration of liquid embolic and/or other bioactive materials useful in the practice of the invention can be readily determined by a skilled operator and it will be understood that any combination of materials, concentration or dosage can be used, so long as it is not harmful to the subject.

It also may be desirable to include one or more radio-opaque materials for use in visualizing the devices in situ. Thus, the injection-molded devices may be coated or mixed with radio-opaque materials such as metals (e.g. tantalum, gold or platinum particles); barium sulfate; bismuth subcarbonate; or the like.

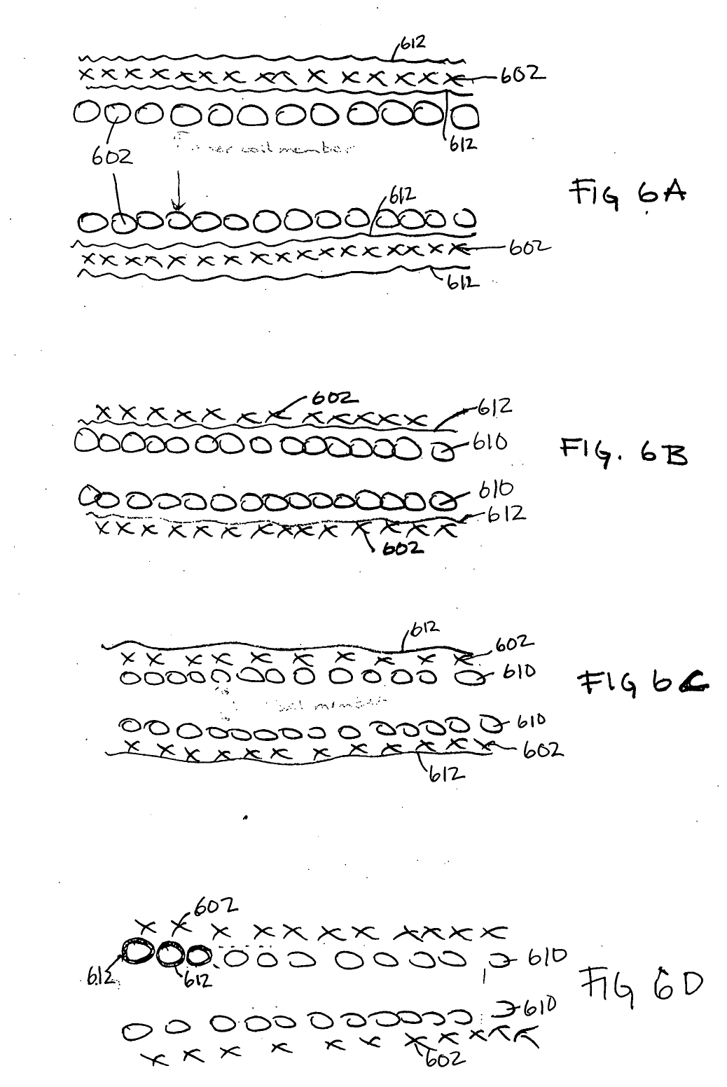

FIG. 6A to 6F are side view cross sections depicting embodiments showing expandable member (602), inner member (610) and additional polymeric element (612). Polymeric element (612) is typically fairly flexible and may be coated onto one or both the expandable member.(602) or inner member (610) or, alternatively, may comprise a separate element from the other members. Furthermore, the polymer element (612) may be loose or may be secured to the inner member (610) and/or expandable member (602) in one or more locations.

FIG. 6A shows an embodiment in which the polymer element (612) is positioned such that it covers the exterior the expandable member (602) and lies between the expandable member (602) and inner member (610). FIG. 6B depicts an embodiment in which the polymer element (612) is positioned between the expandable member (602) and inner member (610). FIG. 6C shows an embodiment in which the polymer element (612) is positioned exterior to the expandable member (602). FIG. 6D depicts an embodiment in which the polymer component is coated onto the inner member (610). Although not depicted, it will be apparent that the polymer component could be coated onto the expandable member (602) instead of, or in addition to, coated on the inner member. When used as a coating, the polymer element (612) may coat all or some of the expandable member (602) and/or inner member (610). The polymer (612) may comprise any of the polymers described herein or combinations thereof and is preferably non-conductive.

Construction

The expandable members and/or inner members described herein may be formed by any suitable method. For example, the members may be formed by winding a wire or filament around a mandrel, using well-known techniques. For example, coil structures may be formed by wrapping or winding a fine filament or wire (102), preferably having a diameter less than about 0.005 inches, preferably 0.00045 to about 0.004 inches, more preferably about 0.0005 to 0.0035 inches, most preferably about 0.001 to 0.003 inches, about a spinning mandrel using well-known coil-manufacturing techniques. A separate end cap (108) or termination piece may be included at the end of the inner member and/or expandable member. The terminator (108) may be a separate piece or a fused portion of the coil or a bit of a filled material such as an epoxy. The end piece may prevent the coil from catching on the interior of the catheter lumen or vessel. However, for all the embodiments shown herein, it is to be understood that a terminator (108) is not required.

Similarly, when the expandable member and/or inner member comprises a non-metal biocompatible material including, for example, Dacron (polyester), polyglycolic acid, polylactic acid, fluoropolymers (polytetrafluoroethylene), Nylon (polyamide), and/or silk, these members may be woven and/or braided from strands of one or more polymers and/or fibers and these strands generally have tensile strength of greater than about 0.10 pounds, for example by weaving fibers or wires over a mandrel much in the same way as the coil member. See, e.g., U.S. Pat. No. 6,458,119. One or more of the members may also be produced by other methods, for example by injection molding or the like. See, e.g., WO 02/096273.

FIG. 7A shows an exemplary an expandable member comprising a braid construction (700) and coil construction (710). The expandable member surrounds the inner member (715). FIG. 7B shows an exemplary expandable member comprising alternating braid (700) and coil constructions (710). FIG. 7C shows an exemplary expandable member comprising alternating braid (700) and coil (710) constructions and where the coil constructions (710) are secured to the inner member (702).

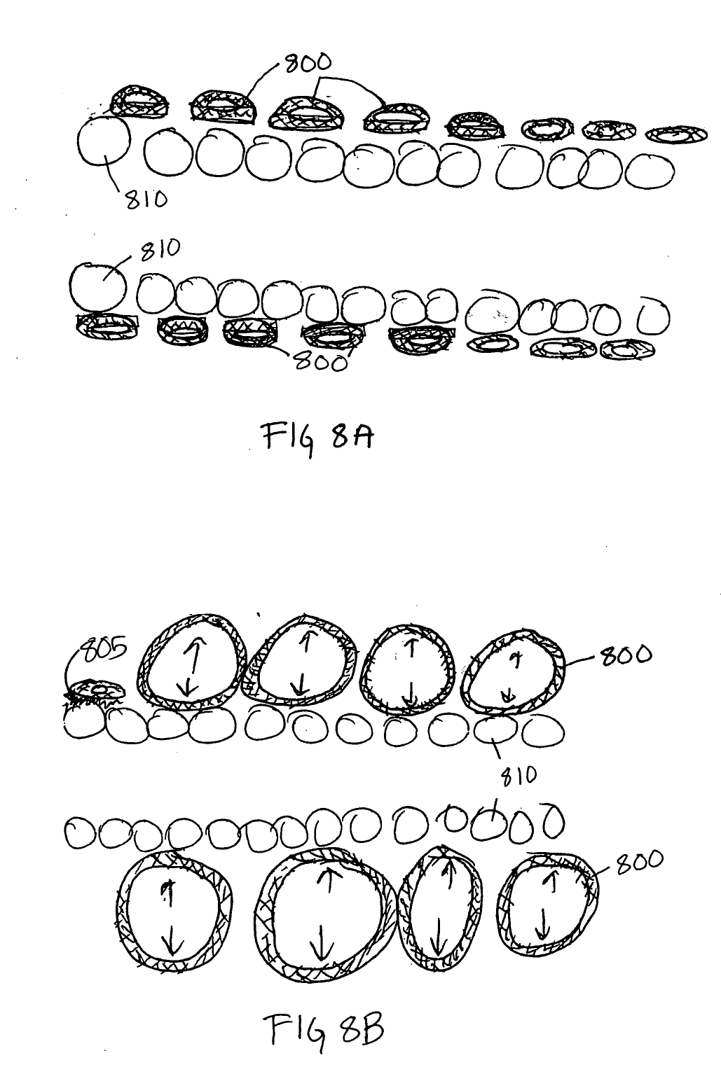

FIG. 8 shows an exemplary device in which the expandable member (800) is constructed by winding a braid over an inner member (810) such that expandable member forms a coil over structure over the inner member (810). FIG. 8A shows this exemplary construction when the braided and wound expandable member (800) is in its restrained form. The expandable member (800) may be restrained by any of the restraining members described herein, for example a catheter, a degradable and/or dissolvable coating, etc. The various dimensions of this exemplary device are also within those given below. FIG. 8B shows expansion of the expandable member (800) after release from the restraining member(s). One or more of the windings of the expandable member (800) may be bonded to the coil (810) in such a way that it does expand (805) upon release from a restraining member.

Braided expandable members can be constructed from a single filament (e.g., mono-filament), multiple filaments (e.g., yam-like materials) or combinations thereof. In certain embodiments, the expandable member is constructed from one or more multifilaments, which may provide high densities upon deployment (e.g., encouraging blood stasis in aneurysm) while maintaining a soft buckling force (less force on aneurysm). In other embodiments, one or more monofilaments are included in the expandable member, as monofilaments may exhibit greater expansion capabilities (e.g., expansion force), thereby potentially facilitating expansion of this member to larger expanded diameters. Braided expandable members, like other constructions, may also include one or more radiopaque materials, for example, radioopaque mono- and/or multi-filaments to aid in visualization during and after deployment.

Generally speaking, when the expandable member comprises a metallic coil that comprises a platinum alloy or a super-elastic alloy such as nitinol, the diameter of the wire used in the production will be in the range of 0.0005 and 0.006 inches. The wire of such diameter is typically then wound into a member having a primary diameter of between 0.01 and 1.25 inches, preferably 0.02 to 0.1 inches. For most neurovascular indications, the preferable diameter is any value between 0.010 to 0.018 inches. Generally, the wire may be of sufficient diameter to provide a hoop strength to the resulting device sufficient to hold the device in place within the chosen body cavity without distending the wall of the cavity and without moving from the cavity as a result of the repetitive fluid pulsing found in the vascular system.

The diameter of the entire device (e.g., inner and expandable members in compressed form) is preferably between about 0.010 and 0.018 inches. The axial length of the expandable member and/or inner member (as restrained) will usually fall in the range of 0.5 to 100 cm, more usually 2.0 to 40 cm. All of the dimensions here are provided only as guidelines and are not critical to the invention. However, only dimensions suitable for use in occluding sites within the human body are included in the scope of this invention.

The inner and/or expandable members may also be secured to each other at one or more locations. For example, to the extent that these members are thermoplastic, they may be melted or fused to other elements of the devices. Alternatively, they may be glued or otherwise fastened. Furthermore, the various elements may be secured to each other in one or more locations. See, e.g., FIG. 7A-C. The expandable member and/or inner member may have a termination piece or section similar in constitution to the analogous portion shown in the Figures. The length of the device maybe 2 mm to 120 cm or even longer. As noted above, the constrained diameter of the expandable member is less than the diameter after deployment.

Thus, the vaso-occlusive devices described herein may be comprised of a variety of material and may take on a variety of shapes and/or configurations. It is further within the scope of this invention that the vaso-occlusive device as a whole or elements thereof (e.g., inner member) comprise shapes or structures other than those shown in the Figures, for examples, spheres, ellipses, spirals, figure-8 shapes, etc. Stretch-resistant configurations can also be designed and manufactured. For example, a fiber material can be threaded through the inside of the inner member and secured to both the proximal and distal end of the device. In addition, the expandable material may be constructed in a way to reduce the likelihood of the inner member stretching. For example, an expandable braid construction may serve to prevent the coil from stretching. In addition, as noted above, any of the devices described herein may further include one or more additional components (described above), which may be, for example, coated onto or otherwise secured to the expandable and/or restraining member(s).

Methods of Use

The embolic compositions described herein are often introduced into a selected site using the procedure outlined below. This procedure may be used in treating a variety of maladies. For instance in the treatment of an aneurysm, the aneurysm itself will be filled (partially or fully) with the compositions described herein. Conventional catheter insertion and navigational techniques involving guidewires or flow-directed devices may be used to access the site with a catheter. The mechanism will be such as to be capable of being advanced entirely through the catheter to place vaso-occlusive device at the target site but yet with a sufficient portion of the distal end of the delivery mechanism protruding from the distal end of the catheter to enable detachment of the implantable vaso-occlusive device. For use in peripheral or neural surgeries, the delivery mechanism will normally be about 100-200 cm in length, more normally 130-180 cm in length. The diameter of the delivery mechanism is usually in the range of 0.25 to about 0.90 mm. Briefly, occlusive devices (and/or additional components) described herein are typically loaded into a carrier for introduction into the delivery catheter and introduced to the chosen site using the procedure outlined below. This procedure may be used in treating a variety of maladies. For instance, in treatment of an aneurysm, the aneurysm itself may be filled with the embolics (e.g. vaso-occlusive members and/or liquid embolics and bioactive materials) which cause formation of an emboli and, at some later time, is at least partially replaced by neovascularized collagenous material formed around the implanted vaso-occlusive devices.

A selected site is reached through the vascular system using a collection of specifically chosen catheters and/or guide wires. It is clear that should the site be in a remote site, e.g., in the brain, methods of reaching this site are somewhat limited. One widely accepted procedure is found in U.S. Pat. No. 4,994,069 to Ritchart, et al. It utilizes a fine endovascular catheter such as is found in U.S. Pat. No. 4,739,768, to Engelson. First of all, a large catheter is introduced through an entry site in the vasculature. Typically, this would be through a femoral artery in the groin. Other entry sites sometimes chosen are found in the neck and are in general well known by physicians who practice this type of medicine. Once the introducer is in place, a guiding catheter is then used to provide a safe passageway from the entry site to a region near the site to be treated. For instance, in treating a site in the human brain, a guiding catheter would be chosen which would extend from the entry site at the femoral artery, up through the large arteries extending to the heart, around the heart through the aortic arch, and downstream through one of the arteries extending from the upper side of the aorta. A guidewire and neurovascular catheter such as that described in the Engelson patent are then placed through the guiding catheter. Once the distal end of the catheter is positioned at the site, often by locating its distal end through the use of radiopaque marker material and fluoroscopy, the catheter is cleared. For instance, if a guidewire has been used to position the catheter, it is withdrawn from the catheter and then the assembly, for example including the absorbable vaso-occlusive device at the distal end, is advanced through the catheter.

Once the selected site has been reached, the absorbable vaso-occlusive device is extruded, for example by loading onto a pusher wire. Preferably, the vaso-occlusive device is loaded onto the pusher wire via a mechanically or electrolytically cleavable junction (e.g., a GDC-type junction that can be severed by application of heat, electrolysis, electrodynamic activation or other means). Additionally, the vaso-occlusive device can be designed to include multiple detachment points, as described in co-owned U.S. Pat. No. 6,623,493 and 6,533,801 and International Patent publication WO 02/45596. They are held in place by gravity, shape, size, volume, magnetic field or combinations thereof.

### EXAMPLES

### Example 1

Two-Component Devices

An inner coil member having a relaxed three-dimensional configuration was made according to standard techniques. An expandable member was made by braiding nitinol and/or polymer wires and heat setting to the desired expanded configuration. Subsequently, the expandable member was cinched over a linearized coil such that the diameter of the nitinol expandable braid was that of the linearized coil and the restrained device loaded into a deployment catheter in the restrained form.

Upon release from the deployment catheter, the coil and nitinol braid assume their relaxed and expanded configurations, respectively.

### Example 2

Actuation of Expansion by Temperature Change

A nitinol braid is constructed to have a superelastic (or shape-memory form) of an expanded diameter using standard heat set techniques. The braid is then cooled below its transformation temperature into its malleable, martinsitic phase. The malleable phase is cinched to a coil such that its diameter is restricted to that of the inner coil member.

During delivery, the device is kept cool, for example using cooled saline. When deployed from the catheter, the braid transitions to the shape-memory form and assumes its expanded configuration. If needed, warmed saline may also be delivered to the target vessel to induce the transition to the expanded form.

Modifications of the procedure and vaso-occlusive devices described above, and the methods of using them in keeping with this invention will be apparent to those having skill in this mechanical and surgical art. These variations are intended to be within the scope of the claims that follow.

1. A vaso-occlusive device comprising a substantially tubular expandable member having a primary axis, wherein the expandable member expands along the primary axis upon release from a restraining member and further wherein the expandable member does not include a hydrogel.

2. The device of claim 1, further comprising an inner member surrounded by the expandable member.

3. The device of claim 1 or claim 2, wherein the expandable member comprises a braid configuration.

4. The device of claim 3, wherein the braid comprises one or more mono-filament.

5. The device of claim 3, wherein the braid comprises one or more multi-filaments.

6. The device of claim 3, wherein the braid comprises one or more mono-filament and one or more multi-filament.

7. The device of claim 2, wherein the inner member comprises a coil having a linear restrained configuration and a relaxed three-dimensional configuration, wherein the coil forms the relaxed three-dimensional configuration upon release from a restraining member.

8. The device of claim 1 or claim 2, further comprising a detachment junction on at least one end of the device.

9. The device of claim 8, wherein the detachment junction comprises an electrolytically detachable end adapted to detach from a pusher by imposition of a current on the pusher.

10. The device of claim 9, wherein the restraining member comprises the detachment junction and one or more ends of the expandable member secured to the detachment junction are released upon the imposition of current.

11. The device of claim 1 or claim 2, wherein the restraining member comprises a deployment catheter.

12. The device of claim 1 or claim 2, wherein the restraining member comprises a degradable or swellable polymer or a water-soluble coating.

13. The device of claim 2, wherein the restraining member comprises an electrolytically detachable member.

14. The device of claim 2, wherein the expandable member is attached to the inner member in at least one location.

15. The device of claim 1, wherein the expandable member comprises a metal.

16. The device of claim 15, wherein the metal is selected from the group consisting of nickel, titanium, platinum, gold, tungsten, iridium and alloys or combinations thereof.

17. The device of claim 16, wherein the metal is nitinol.

18. The device of claim 1, wherein the expandable member comprises a polymer selected from the group consisting of poly(ethyleneterephthalate), polypropylene, polyethylene, polyglycolic acid, polylactic acid, nylon, polyester, fluoropolymer, and copolymers or combinations thereof.

19. The device of claim 15, wherein the expandable member further comprises one or more polymers.

20. The device of claim 19, wherein the polymer is selected from the group consisting of poly(ethyleneterephthalate), polypropylene, polyethylene, polyglycolic acid, polylactic acid, nylon, polyester, fluoropolymer, and copolymers or combinations thereof.

21. The device of claim 2, wherein the inner member comprises a coil comprising a metal selected from the group consisting of platinum, palladium, rhodium, gold, tungsten and alloys thereof.

22. The device of claim 2, wherein the inner member comprises a coil comprising a stainless steel or super-elastic metal alloy.

23. The device of claim 22, wherein the coil member comprises nitinol.

24. The device of claim 2, further comprising a polymer element positioned between the expandable member and the inner member.

25. The device of claim 2, further comprising a polymer element coated onto the expandable member or the inner member.

26. The device of claim 2, further comprising an additional component coated onto the expandable member or the restraining member.

27. The device of claim 26, wherein the additional component is bioactive.

28. A method of occluding a body cavity comprising introducing a vaso-occlusive device according to claim 1 into the body cavity.

29. A method of occluding a body cavity comprising introducing a vaso-occlusive device according to claim 2 into the body cavity.

30. The method of claim 28 or claim 29, wherein the body cavity is an aneurysm.
